(19) United States
(12) Patent Application Publication
(10) Pub. No.: US 2007/0073262 A1
(43) Pub. Date: Mar. 29, 2007

(54) Absorbent article and method of making same

(76) Inventors: Kimberly Babusik, Wenonah, NJ (US); Dennis McVey, Lancaster, PA (US); Jocelyn P. Rice, Philadelphia, PA (US)

Correspondence Address: RATNERPRESTIA, P O BOX 980, VALLEY FORGE, PA, 19482-0980, US

(21) Appl. No.: 11/236,994

(22) Filed: Sep. 28, 2005

(51) Int. Cl. A61F 13/15

(52) U.S. Cl. 604/396; 604/385.3

(57) ABSTRACT

The invention provides a disposable protective undergarment including front and back panels, each panel having a waist end, and a crotch end longitudinally spaced from the waist end, and two sides, the crotch ends of the front panel and back panel having a substantially straight edge and a substantially convex curved edge, respectively, with a crotch insert bridging the gap therebetween. The invention further provides a method of manufacturing disposable protective undergarments, including forming front and back panel portions of the undergarment from a continuous web, each front and back panel portion having a waist end and a crotch end longitudinally spaced from the waist end and having a substantially straight edge, removing a portion of the crotch end of the back panel to form a substantially convex curved edge, and fixing a crotch insert to crotch ends of the front and back panel portions to bridge a gap therebetween.

### FIELD OF THE INVENTION

This invention relates generally to disposable absorbent undergarments, e.g., disposable underwear, adult and youth briefs, and training pants for children. More specifically, this invention relates to articles of that type that can be manufactured efficiently and economically and which exhibit protection from leakage, good fit and comfort.

### BACKGROUND OF THE INVENTION

Absorbent articles such as disposable diapers, training pants, adult incontinence garments, and the like are known, their major function being to absorb and contain body exudates. Such articles are thus intended to prevent the soiling, wetting, or other contamination of clothing or other articles, such as bedding, that come into contact with the wearer. In the case of disposable protective undergarments, a variety of designs and manufacturing techniques have been developed to maximize performance and comfort to the wearer and to obtain a high degree of efficiency in manufacturing while minimizing the amount of waste material, thereby keeping production costs as low as possible. However, many of the current designs and manufacturing methods have been unable to achieve an optimum balance between these desirable, yet often diverging goals. While many developments have been made in the art of disposable absorbent undergarments, there remains a need for further improvements in both design and manufacturing.

### SUMMARY OF THE INVENTION

The invention, according to one exemplary embodiment, provides a three-component disposable protective undergarment including a front panel, a back panel and a crotch insert. In one aspect of the invention, the front panel has a waist end, a crotch end longitudinally spaced from the waist end and having a substantially straight edge, and two sides extending between the waist end and the crotch end. The back panel also includes a waist end, a crotch end longitudinally spaced from the waist end with the crotch end having a substantially convex curved edge, and two sides extending between the waist end and the crotch end. The crotch end of the back panel is longitudinally separated from the crotch end of the front panel by a gap, and the sides of the back panel are fixed to respective sides of the front panel to form a continuous waist, which is made up of the waist ends of the front and back panels. The crotch insert has a front end fixed to the front panel, a back end fixed to the back panel, and two sides extending between the front end and the back end. The crotch insert, therefore, bridges the gap between the crotch end of the front panel and the crotch end of the back panel. The portions of the crotch end of the front panel, the crotch end of the back panel, and the sides of the crotch insert together define leg openings.

In another aspect, the invention provides a method of manufacturing a disposable protective undergarment. The method includes the step of forming the front panel and back panel portions of the undergarment from a continuous web of material. In this aspect, each front and back panel portion has a waist end and a crotch end longitudinally spaced from the waist end and having a substantially straight edge. The method further includes the step of removing a portion of the crotch end of the back panel portion to form a substantially convex curved edge. In addition, the method also includes the step of fixing a front end of a crotch insert to the front panel portion and a back end of the crotch insert to the back panel portion to bridge a gap between the front and back panels.

In yet another aspect, the present invention provides a protective undergarment having a front panel, a back panel and a crotch insert. According to this aspect, the front panel has a waist end having a substantially straight edge, a crotch end longitudinally spaced from the waist end and having a substantially straight edge, and two sides extending between the waist end and the crotch end. The crotch end of the front panel according to this embodiment is substantially parallel to the waist edge of the front panel. The back panel of the protective undergarment according to this aspect includes a waist end, a crotch end longitudinally spaced from the waist end and having a substantially convex curved edge, and two sides extending between the waist end and the crotch end. In this aspect, the crotch end of the back panel is longitudinally separated from the crotch end of the front panel by a gap and the sides of the back panel are fixed to respective sides of the front panel to form a continuous waist. The continuous waist according to this aspect is comprised of the waist end of the back panel and the waist end of the front panel. In addition, the undergarment includes a crotch insert having a front end fixed to the front panel, a back end fixed to the back panel, and two sides extending between the front end and the back end, the crotch insert thereby bridging the gap between the crotch ends of the front and back panels. Together with the crotch insert, the crotch ends of the front and back panels thus define leg openings according to this aspect of the invention.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a plan view of an exemplary embodiment of a disposable absorbent undergarment in a partially unassembled, stretched flat state in which the bodyside surface of the absorbent undergarment faces the viewer.

FIG. 2 is a front view of an exemplary embodiment of the absorbent undergarment in its assembled state.

FIG. 3 is a cross-sectional view of the absorbent composite of the absorbent protective undergarment of FIG. 1 (along line 3-3), according to one aspect of this invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

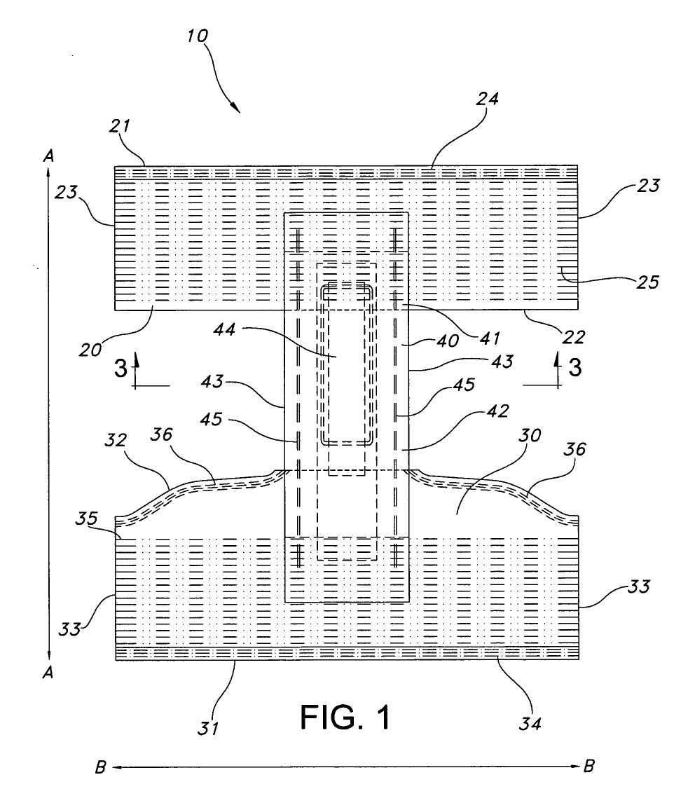

Referring generally to the drawing, FIG. 1 illustrates an embodiment in which the present invention is shown in a partially unassembled, stretched flat state, the bodyside surface of the disposable absorbent undergarment facing the viewer. The arrows A-A generally indicate a longitudinal dimension of the undergarment in the partially unassembled state. This longitudinal orientation corresponds to the longitudinal orientation of the crotch portion of the undergarment, as will be described later. Also, the arrows B-B generally indicate width dimension of the undergarment in the partially unassembled state. The direction B-B also corresponds to the machine direction optionally used to make components of the undergarment. More specifically, front and back panels of the undergarment (described below) are optionally formed from a continuous web or continuous webs that are directed along a machine direction parallel to B-B.

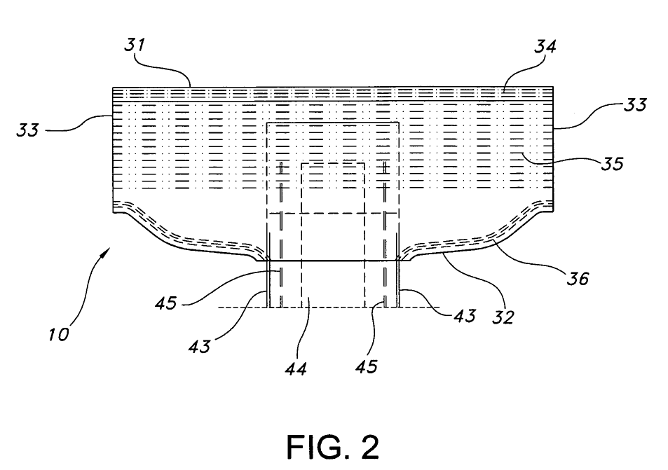

With reference to FIG. 1, a three-component disposable protective undergarment 10 is provided, comprising a front panel 20, a back panel 30, and a crotch insert or panel 40. Fully assembled, as shown in FIG. 2, the disposable absorbent undergarment 10 forms a pants-shaped chassis.

As shown in FIG. 1, the front panel of the disposable protective undergarment 10 includes a substantially straight waist end 21 along a transverse direction, a substantially straight crotch end 22 longitudinally spaced from the waist end 21 and running in a transverse direction, and two substantially straight sides 23 that extend between the waist end 21 and the crotch end 22. Again, the term “longitudinally” as used herein refers to the direction along line A-A, while the term “transverse” refers to the direction along line B-B, as shown in FIG. 1.

The back panel of the disposable protective undergarment 10 according to one aspect of the invention, includes a substantially straight waist end 31 along a transverse direction, a crotch end 32 longitudinally spaced from the waist end 31 and extending in a transverse direction, and two substantially straight sides 33 extending between the waist end 31 and the crotch end 32, where the crotch end 32 of the back panel 30 has substantially convex curved edges separated by a substantially straight crotch end portion where the crotch panel 40 is fixed to the back panel 30. The back panel 30 is separated longitudinally from the front panel 20 by a gap therebetween.

As illustrated in FIG. 1, crotch insert 40 has a front end 41, a back end 42 longitudinally spaced from the front end 41, and two sides 43 extending between the front end and the back end. The front end 41 is fixed to the front panel 20 and the back end 42 is fixed to the back panel 30 thereby bridging the gap between the crotch end 22 of the front panel 20 and the crotch end 32 of the back panel 30. The crotch panel 40, as shown in FIG. 1, has a generally rectangular shape and includes front end 41, back end 42 and sides 43. The crotch panel may also have other shapes, for example an oval shape. In an embodiment of the invention, the crotch insert 40 may include an absorbent composite 44 attached to or otherwise associated with a central portion of the crotch panel 40.

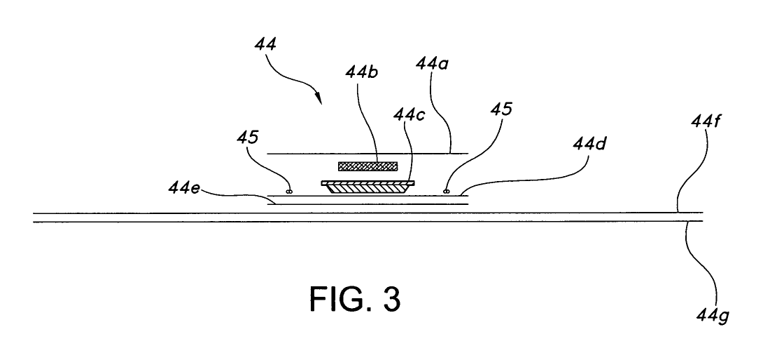

The absorbent composite of an exemplary embodiment of the disposable absorbent undergarment of the present invention can be as shown in FIG. 3. This embodiment includes a multilayer absorbent composite 44 having a body side (top as shown in FIG. 3) and a garment side (bottom as shown in FIG. 3). As shown in the cross-sectional view, beginning from bodyside and continuing to the garment side, the absorbent composite 44 includes a cover 44a adjacent an acquisition/distribution layer (ADL) 44b, which is disposed adjacent an absorbent core 44c, which is in turn adjacent a backsheet 44d. Adjacent the bodyside of the backsheet are disposed an insert cover 44e, which lies above inner cover 44f and outer cover 44g. Inner cover 44f and outer cover 44g correspond to inner and outer panels that, in certain embodiments, form front 20 and back 30 panels, as described in more detail below. In another embodiment, the ADL may comprise two separate layers including an acquisition layer and a distribution layer. While one exemplary embodiment of a crotch panel and absorbent composite are shown for purposes of illustration, a wide variety of panels and composites are optionally used within the scope of this invention.

Referring to the embodiment illustrated in FIG. 3, however, the cover 44a of the absorbent composite 44 is a liquid pervious layer which allows liquid to easily penetrate the cover to the layers beneath. Suitable materials for the liquid-permeable cover 44a include non-woven spunbond polypropylene of 15 gsm available from Avgol of Holon, Israel, wherein the non-woven material is zone coated, with its longitudinal central area being hydrophilic and its two longitudinal side areas being hydrophobic. The cover 44a may also be formed of an apertured three dimensional film or a combination of such film and a non-woven material. In accordance with an exemplary embodiment of the undergarment 20, the cover 44a is of the same size and shape as the backsheet 44d and is disposed thereover. The cover 44a and the backsheet 44d may be wider than the other layers such that the cover 44a and backsheet 44d are directly connected to each other, thereby sandwiching the ADL 44b and absorbent core 44c therebetween. The cover 44a is glued to the backsheet 44d with the ADL 44b, and the absorbent core 44c interposed therebetween.

In the exemplary embodiment shown in FIG. 3, an ADL 44b is used between the absorbent core 44c and the cover 44a. The ADL 44b is located over the thickened portion of the absorbent core 44, i.e., the portion where the fluid insult is expected to be initiated. The ADL 44b serves to manage, transport, accommodate and/or direct high volumes and flow rates of fluid into the core 44c.

The ADL 44b can be thru-air bonded/carded web, a spunbond bi-component non-woven web, a web of crosslink cellulosic fibers, apertured 3D (three dimensional) film or the like. One particularly suitable material is a thru-air bonded bi-component with a fast finish surfactant available from PGI Non-Wovens of Landisville, N.J., which has an overall basis weight of 40 gsm. The bi-component fibers are made of a polypropylene inner core and a polyethylene outer sheath. The ADL 44b may be adhesively secured in place by any suitable construction adhesive, such as 34-373A adhesive available from National Starch and Chemical of Bridgewater, N.J.

Interposed between the ADL 44b and the backsheet 44d is the absorbent core 44c. The absorbent core 44c, according to one embodiment, is made from untreated pulp and contains superabsorbent polymer. The absorbent composite 44, as shown in the embodiment illustrated in FIG. 1, lies transversely centered in the crotch insert 40, but is off-set toward the front panel in the longitudinal direction of the crotch insert, such that a portion of the absorbent composite 44 overlaps the belly bands 25 of the front panel 20.

The absorbent core 44 is disposed on top of the backsheet 44d. In the embodiment shown herein, the absorbent composite 44 is of a generally rectangular shape. If desired, it may have a central portion of increased thickness as compared to its side marginal portions. The thickened central portion of the absorbent core 44c may taper from the front to the rear. These structural features of the core 44c and its location ensures that the maximum thickness of the core is located at the anatomical position at which the urine insult from the wearer will be initiated.

The core 44c can be formed of any suitable material(s), such as an airlaid composite, containing pulp, superabsorbent particulates and/or fibers, and binders. The binders may be chemical or thermal.

In accordance with one exemplary embodiment, the core 44c is made up of mainly cellulosic fibers, e.g., wood pulp fluff made of up bleached sulfate wood pulp containing soft wood fibers, such as that available from International Paper of Tuxedo, N.Y., co-mingled with hydrogel polymer particulates (known as Super Absorbent Polymer or “SAP”) such as cross-linked polyacrylate IM-3900 available from BASF Corporation of Charlotte, N.C. If desired, these materials may be optionally wrapped in tissue. The amount of each absorbent material and SAP/fluff ratio depends on the size of the protective underwear, whether it is used for children (e.g., training pants) or for adults, and whether or not a transfer or fluid acquisition layer component is to be included in the absorbent composite 44. In this regard, the ADL 44b as shown herein may be optionally omitted from the undergarment 10.

In order to prevent the egress of fluid out through the non-woven sheets of the chassis, the crotch insert 40 also includes the heretofore identified backsheet 44d. The backsheet 44d is interposed between the core 44c and the chassis and is formed of a liquid impervious material, e.g., a microporous polyfilm which is vapor permeable (breathable). In the embodiment shown herein, the backsheet 44d is of the same generally rectangular shape and size as the cover 44a and is located in the crotch insert 40 of the undergarment 10. In accordance with one exemplary embodiment, the backsheet 44d is formed of a film of 0.5 mil polypropylene available from Pliant Corporation of Williamsburg, Va.

The crotch insert 40 of the absorbent garment also includes two sets of elastic threads 45 comprising at least one elastic strip each. The elastic threads 45 extend along the longitudinal direction of the crotch insert 40 for a distance along each side 43 of the crotch insert 40 such that each elastic thread 45 also overlaps the belly band regions 25 of the front 20 and back 30 panels. The elastic threads 45 are sandwiched between the cover 44a and backsheet 44d and are attached using construction adhesive, without overlap of the other insert layers illustrated in FIG. 3. In addition, the front 20 and back 30 panels and crotch insert 40 of the undergarment 10 include an inner cover 44f and an outer cover 44g also as illustrated.

While the invention includes the various embodiments described above, other embodiments of the invention may also include features as shown and described in U.S. patent application Ser. No. 10/617,218 to Glaug et al., filed on Jul. 10, 2003, which application is incorporated herein by reference. Each of the embodiments of the present invention is expected to provide equal or better functional performance and customer acceptance at a lower cost as compared to conventional protective undergarments. In particular, equal or improved performance is expected with respect to characteristics such as leakage control, coverage in the front, coverage in the back, fit at the waist, fit at the legs, overall fit, resident comfort, and ease of putting on and removing the undergarment.

When fully assembled, as shown in FIG. 2, the sides 23 of the front panel 20 are fixed to the respective sides 33 of the back panel 30 so as to form a continuous waist comprising the waist end 21 of the front panel and the waist end 31 of the back panel. Further, the crotch end 22 of the front panel 20 and the crotch end 32 of the back panel 30, along with the sides 43 of the crotch insert together define leg openings. Each leg opening therefore has a linear edge at the crotch end 22 of the front panel 20; a convex edge at the crotch end 32 of the back panel 30 (along the convex curved edge); and an intermediate, substantially straight edge along the sides 43 of the crotch panel 40. The undergarment chassis is thus configured such that when it is worn it covers a portion of the wearers legs beneath the buttocks, while a front portion of the wearer's leg where the leg meets the torso is exposed. The undergarment therefore allows increased exposure of the front portion of the wearer's legs without sacrificing coverage of the wearer's buttocks.

The material forming the front 20 and rear 30 panels of the chassis can be any suitable material used in disposable absorbent articles. In the interest of wearing comfort, it is preferably a non-woven, cloth-like breathable material, such as spunbond/meltblown/spunbond polypropylene at 15 gsm available from Avgol of Holon, Israel. The material making up the chassis may be a polylaminate, e.g., polyfilm bonded to a non-woven material. The adhesive for securing the webs together with the elastic fibers interposed therebetween, may be a construction adhesive, such as that available from National Starch and Chemical Company of Bridgewater, N.J.

Referring again to FIG. 1, the front panel 20 has a generally rectangular shape and includes a plurality of waist elastics 24 in the top portion of the panel 20 that lie along the entire transverse length of the front panel 20. These waist elastics may be made from elastic filaments. The front panel 20 also includes a plurality of elastic threads or filaments to form belly bands 25 that lie along the entire transverse length of the front panel 20 and that lie beneath the top edge of the front panel 20. Each belly band may be comprised of an elastic filament as is known in the art and may be separated from the next adjacent belly band by a uniform distance. In at least one embodiment of the invention, there are three waist elastics and 17 belly bands.

Referring to the same illustration, mirroring the front panel 20, the back panel 30 also includes a plurality of waist elastics 34, which also may be made from elastic filaments or threads as is known in the art. Likewise, the back panel 30 includes a plurality of elastic threads or filaments forming belly bands 35 that are similar to that of the front panel 20. In at least one embodiment of the invention, there are three waist elastics and 15 belly bands. In addition, the back panel 20 includes curved leg elastic 36. The curved leg elastic 36 runs from the transverse length of the back panel 30 such that the leg elastic 36 conforms to the shape of the convex curved edge of the back panel 30. However, the leg elastic is not continuous, but rather split by a gap (resulting from the removal of a central portion of the leg elastic during manufacturing, as described below) within which the crotch insert 40 is located.

Referring generally to the figures, a method of manufacturing the disposable protective undergarment 10 will now be described. Generally, the first step includes forming front and back panel portions of the disposable absorbent undergarment from at least one continuous web of material. Each of the front and back panel portions have a waist end and a crotch end that are longitudinally spaced apart and have substantially straight edges. In another step, a portion of the crotch end of the back panel is removed to form a substantially convex curved edge. In yet another step, the front end of the crotch insert is fixed to the front panel while the back end of the crotch insert is fixed to the back panel. The crotch insert thus bridges the gap between the front and back panels. Though the manufacturing steps described herein are preferable performed in the order described, they can be performed in any order, depending upon the equipment and processes selected and the preferences of the manufacturer.

Describing in further detail one exemplary embodiment of the manufacturing method, the method involves several steps performed in a substantially continuous operation. In one aspect of the invention, substrates are provided that are used to form inner and outer layers of the front and back panels. The continuous web that makes up the front and back inner panels is loaded into a processing machine. As the inner layer material moves through the machine, the web is cut into two web portions along a straight line longitudinally extending in the machine direction (direction B-B shown in the figures), the two webs comprising the web portions that form the back panel and the front panel. Each of these front and back inner panels have a waist end and a crotch end longitudinally spaced in direction A-A from the waist end and have a substantially straight edge. Similarly, the outer layer is also loaded into the machine and is likewise cut into two webs.

The inner web portions are processed through a series of rollers after which elastic threads, which may include waist elastic and belly bands, are applied. The elastic threads, which may be applied in a pretensioned state, are then coated with glue by strand coating glue heads. Then, the inner front panel web portion is “married-up” with the outer front panel web portion as the inner and outer webs are combined by a combining drum, thus sandwiching the elastic threads therebetween. The term “married up” is used herein to mean that the inner and outer web portions are substantially aligned and at least partially joined together to form a single panel.

After being combined by the combining drum, the front panel passes through a roller and under the insert attachment glue head at which point adhesive is applied to the front panel by a speed coat slot nozzle glue head. The adhesive is applied in a rectangular region so as to correspond to the overlap between the crotch insert and the front panel, as discussed in more detail below.

Next, after the back panel inner web portion passes through the series of rollers, elastic adhesive is applied by a speed coat slot nozzle glue head. The web portion is processed through a roller, after which elastic threads which may be pretensioned, are applied. In one aspect, these elastic threads in addition to waist elastic and belly bands, include leg elastic threads that conform to the convex curved contour of the crotch end of the back panel. The back panel web is then married-up with the back panel outer web portion by the combining drum, thereby sandwiching the elastic threads therebetween.

After the back panel inner and outer webs are married up, a thin strip of the crotch end of the back panel is removed. The strip that is removed includes a portion of the web material and may also include portions of the leg elastic threads disposed in the area where the crotch insert is to be attached on the back panel of the disposable absorbent undergarment. In other words, the leg elastic threads or filaments are optionally applied to the back panel along a continuous length. The removal of a portion of the crotch end of the back panel may therefore remove a portion of the leg elastics in the location at which the crotch section is fixed to the back portion of the garment.

The step of removing a portion of the crotch end of the back panel portion therefore optionally involves retaining the crotch end of the back panel in a crotch insert fixation region. This is optionally accomplished by removing portions of the crotch end of the back panel on opposite sides of the crotch insert fixation region. Though this step can be performed in any order, the step of removing a portion of the crotch end of the back panel portion is advantageously performed before the step of fixing the front end of the crotch insert to the front panel portion and the back end of the crotch insert to the back panel portion.

Adhesive is next applied by a speed coat slot nozzle glue head. The adhesive is applied such that the entire area that the crotch insert will cover on the front and back panel is coated. While the adhesive is applied so as to ensure that as much of the peripheral edges of the crotch insert are adhered to the front and back panels, the adhesive is also applied so as to minimize adhesive leakage beyond the overlapping regions of the crotch insert with the front and back panels, respectively, from overspray or over application of the adhesive. Any such leakage may result in unintended adhesion of portions of the absorbent undergarment during the manufacturing and packaging processes.

After the adhesive is applied, the front and back ends of the crotch insert are placed on the corresponding front and back panels of the diaper. Upon drying, the adhesive holds the crotch insert firmly in place along the entire periphery where it overlaps the front and back panels of the absorbent undergarment.

In an aspect of the invention, the crotch insert of the absorbent undergarment is then folded so as to align the waist end and opposing sides of the front panel with the waist end and opposing sides of the back panel. By connecting the front and the back panel along their sides, the arrangement forms a pants-shaped chassis having a waist opening and two leg openings. Individual products are then cut from the continuous web at the side connections of the panels.

Alternatively, the method may not include a folding step. In this case, the undergarment is not folded and the sides of the undergarment (and consequently the front and back panels) are not connected together during the manufacture of the undergarment. In such an embodiment, the undergarment optionally includes fasteners, such as adhesive tapes, hook and loop fasteners, or other conventional fasteners that are used in conventional disposable diapers that can be secured together at the time of use to form a pants-shaped chassis.

While preferred embodiments of the invention have been shown and described herein, it will be understood that such embodiments are provided by way of example only. Numerous variations, changes and substitutions, including but not limited to the materials that make up the various components of the absorbent protective undergarment, will occur to those skilled in the art without departing from the spirit of the invention. Accordingly, it is intended that the appended claims cover all such variations as fall within the spirit and scope of the invention.

What is claimed:

1. A protective undergarment comprising:
a front panel having a waist end, a crotch end longitudinally spaced from said waist end and having a substantially straight edge, and two sides extending between said waist end and said crotch end;
a back panel having a waist end, a crotch end longitudinally spaced from said waist end and having a substantially convex curved edge, and two sides extending between said waist end and said crotch end, said crotch end of said back panel being longitudinally separated from said crotch end of said front panel by a gap, and said sides of said back panel being fixed to respective sides of said front panel to form a continuous waist comprising said waist end of said back panel and said waist end of said front panel; and
a crotch insert having a front end fixed to said front panel, a back end fixed to said back panel, and two sides extending between said front end and said back end, said crotch insert bridging said gap between said crotch end of said front panel and said crotch end of said back panel;
wherein portions of said crotch end of said front panel, said crotch end of said back panel, and said sides of said crotch insert together define leg openings.

2. The undergarment of claim 1 further comprising an absorbent composite associated with said crotch insert.

3. The undergarment of claim 2 wherein said absorbent composite comprises a coversheet overlaying an acquisition layer, a distribution layer, an absorbent core and a backsheet.

4. The undergarment of claim 3 wherein said acquisition layer and said distribution layer are combined into one layer.

5. The undergarment of claim 1 wherein said two sides of said front panel are adhered to said two opposing sides of said back panel so as to form a pants-shaped chassis having a waist opening and two leg openings.

6. The undergarment of claim 1 wherein said two sides of said front panel are releasably attached to said two sides of said back panel so as to form a pants-shaped chassis having a waist opening and two leg openings.

7. The undergarment of claim 1 wherein said crotch insert includes elastic threads extending longitudinally along said sides of said crotch insert.

8. The undergarment of claim 7 wherein said elastic threads of said crotch insert are sandwiched between said coversheet and said backsheet on opposing sides of said crotch insert.

9. The undergarment of claim 1 further comprising a first set of elastic threads extending along a transverse length of said front and said back panels corresponding to a waist section of said undergarment.

10. The undergarment of claim 9 wherein said first set of elastic threads are made from elastic filaments or threads.

11. The undergarment of claim 9 further comprising a second set of elastic threads extending along a transverse length of said front and said back panels corresponding to a belly section of said undergarment.

12. The undergarment of claim 11 wherein said second set of elastic threads are made from elastic filaments or threads.

13. The undergarment of claim 11 further comprising a third set of elastic threads extending transversely along said crotch end of said back panel so as to conform to said substantially convex curved shape of said back panel.

14. The undergarment of claim 1, wherein said waist end of said front panel has a substantially straight edge and said substantially straight edge of said crotch end of said front panel is substantially parallel to said substantially straight edge of said waist end.

15. The undergarment of claim 14, wherein said leg openings are defined by portions of said substantially straight edge of said crotch end of said front panel, said substantially convex curved edge of said crotch end of said back panel, and said sides of said crotch insert.

16. A method of manufacturing a protective undergarment comprising the steps of:
forming from at least one continuous web of material front panel and back panel portions each having a waist end and a crotch end longitudinally spaced from the waist end and having a substantially straight edge;
removing a portion of the crotch end of the back panel portion to form a substantially convex curved edge; and
fixing a front end of a crotch insert to the front panel portion and a back end of the crotch insert to the back panel portion to bridge a gap between the front and back panel portions.

17. The method of claim 16 further comprising the step of:
folding the crotch insert inwardly so as to align the waist end of the front panel portion with the waist end of the back panel portion.

18. The method of claim 16 further comprising the step of:
connecting the front panel portion and the back panel portion.

19. The method of claim 18 said connecting step comprising connecting the front panel portion and the back panel portion along side regions extending between the crotch ends and the waist ends of the front and back panel portions.

20. The method of claim 19 further comprising the step of:
separating individual products by cutting the front panel portion and the back panel portion along the side regions.

21. The method of claim 16 wherein the forming step further comprises the step of:
cutting a continuous first web of material into web portions along a straight line extending in a machine direction to form a front inner panel and a back inner panel, each of the front and back inner panels having a waist end and a crotch end longitudinally spaced from the waist end and having a substantially straight edge.

22. The method of claim 21 wherein the forming step further comprises the step of:
cutting a continuous second web of material into webs portions along a straight line extending in the machine direction to form a front outer panel and a back outer panel, each of the front and back outer panels having a waist end and a crotch end longitudinally spaced from the waist end and having a substantially straight edge.

23. The method of claim 22 further comprising the step of:
applying a plurality of elastic threads or filaments to the front inner panel or the front outer panel and to the back inner panel or the back outer panel.

24. The method of claim 23 further comprising the steps of:
marrying up the front inner panel with the front outer panel so as to sandwich the plurality of elastic threads or filaments between the front inner panel and the front outer panel; and
marrying up the back inner panel with the back outer panel so as to sandwich the plurality of elastic threads or filaments between the back inner panel and the back outer panel.

25. The method of claim 16, said step of removing a portion of the crotch end of the back panel portion comprising retaining the crotch end of the back panel in a crotch insert fixation region.

26. The method of claim 25, said step of removing a portion of the crotch end of the back panel portion comprising removing portions of the crotch end of the back panel on oppose sides of the crotch insert fixation region.

27. The method of claim 26, said step of removing a portion of the crotch end of the back panel portion being performed before the step of fixing the front end of the crotch insert to the front panel portion and the back end of the crotch insert to the back panel portion.

28. A protective undergarment comprising:
a front panel having a waist end having a substantially straight edge, a crotch end longitudinally spaced from said waist end and having a substantially straight edge that is substantially parallel to said edge of said waist end, and two sides extending between said waist end and said crotch end;
a back panel having a waist end, a crotch end longitudinally spaced from said waist end and having a substantially convex curved edge, and two sides extending between said waist end and said crotch end, said crotch end of said back panel being longitudinally separated from said crotch end of said front panel by a gap, and said sides of said back panel being fixed to respective sides of said front panel to form a continuous waist comprising said waist end of said back panel and said waist end of said front panel; and
a crotch insert having a front end fixed to said front panel, a back end fixed to said back panel, and two sides extending between said front end and said back end, said crotch insert bridging said gap between said crotch end of said front panel and said crotch end of said back panel;
wherein portions of said crotch end of said front panel, said crotch end of said back panel, and said sides of said crotch insert together define leg openings.
